(19) United States
(12) Patent Application Publication
(10) Pub. No.: US 2007/0270606 A1
(43) Pub. Date: Nov. 22, 2007

(54) PROCESS FOR THE MANUFACTURE OF C1 -C4 ALKYL (METH)ACRYLATES

(75) Inventors: Alain Riondel, Forbach (FR); Frederic Sandre, Saint-Avold (FR)

Correspondence Address: ARKEMA INC.;PATENT DEPARTMENT - 26TH FLOOR, 2000 MARKET STREET, PHILADELPHIA, PA, 19103-3222, US

(73) Assignee: Arkema France, Puteaux (FR)

(21) Appl. No.: 11/749,357

(22) Filed: May 16, 2007

(30) Foreign Application Priority Data
May 18, 2006 (FR) 06.51820

(51) Int. Cl. C07C 69/52

(52) U.S. Cl. 560/205

(57) ABSTRACT

The invention relates to an improved process for the manufacture of C₁-C₄ alkyl (meth)acrylates by a direct esterification of (meth)acrylic acid by the corresponding alcohol in the presence of sulphuric acid, phenothiazine being used as polymerization inhibitor. The said process comprises a stage of recovering in value the heavy byproducts generated during this manufacture which consists of a distillation at a relatively low temperature and under an inert atmosphere, followed by a catalytic cracking under an inert atmosphere.

### TECHNICAL FIELD

The present invention relates to the manufacture of C₁-C₄ alkyl (meth)acrylates by direct esterification of (meth)acrylic acid by the corresponding alcohol, this reaction being catalysed by sulphuric acid. A more particular subject-matter of the invention is an improved process for the manufacture of C₁-C₄ alkyl (meth)acrylates comprising a stage in which the heavy byproducts generated during this manufacture are recovered in value.

### PRIOR ART AND TECHNICAL PROBLEM

The problems which are posed during the manufacture of C₁-C₄ alkyl (meth)acrylates will now be set out, for convenience, on the basis of the example of the direct esterification of acrylic acid by butanol. These problems and the solution provided by the invention are the same in the case of the use, on the one hand, of methacrylic acid and, on the other hand, of alcohols other than butanol.

The industrial process for the manufacture of butyl acrylate, such as that described, for example, in European Patent EP 609 127 of the Applicant Company, consists in esterifying acrylic acid by excess butanol in the presence of sulphuric acid. The reaction mixture, at the end of the reaction, comprises butyl acrylate, excess butanol, residual acrylic acid, butyl hydrogen sulphate, traces of sulphuric acid, the stabilizers conventionally used to inhibit polymerization reactions and various impurities resulting from side reactions.

Mention may be made, among the byproducts generated according to side reactions, of light products, such as butyl acetate, butyl propionate, dibutyl ether, isobutyl acrylate or dibutyl maleate. As side reactions resulting in the formation of heavy products, butanol and unreacted acrylic acid add to the double bond of the butyl acrylate already formed (Michael addition) to form, on the one hand, butyl butoxypropionate (subsequently denoted BBP) and butyl hydroxypropionate (subsequently denoted BHP) and, on the other hand, butyl acryloyloxypropionate (subsequently denoted AA/BuA). Polyaddition or the formation of mixed compounds is also possible. One characteristic of the heavy byproducts is that their boiling point occurs above the boiling points of acrylic acid, butanol and butyl acrylate formed.

In the conventional process, the esterification reaction is followed by various purification stages, generally carried out continuously, which result in the recovery of the purified butyl acrylate. One of these stages consists in particular in distilling off the butanol and the light byproducts. The butanol can thus be recycled to the esterification reaction.

The final stage of purification of butyl acrylate consists subsequently in conveying the mixture, comprising the ester freed from the light products, to a distillation column from where it exits at the top, purified from the heavy byproducts, which for their part are found in the distillation column bottom. The heavy byproducts thus generated in the manufacture of butyl acrylates present a treatment problem as they have to be disposed of in the end, for example by incineration. They have a harmful effect on the recovery of the butyl acrylate as, even if they are removed continuously from the reaction system, they accumulate in this system, which can make it increasingly difficult to isolate the butyl acrylate. In addition, they present a problem of undesirable losses of acrylic acid and butanol starting materials and consequently result in a considerable loss in output.

Various solutions have been provided in order to solve the abovementioned problems, in particular the conversion, by an appropriate treatment, of these byproducts into products which can be recycled, such as butyl acrylate and butanol. The various routes envisaged have formed the subject-matter of various patent applications.

The document FR 2 194 681 describes the thermal conversion of the mixture of oligomers which forms the residue deposited in a distillation reactor in which an alcohol is esterified by acrylic acid to give the corresponding acrylic ester. The residue additionally comprises the esterification catalyst, such as an alkyl- or arylsulphonic acid, and the polymerization inhibitor. The residue is heated at a temperature of at least 180° C. with an effective amount of a vinyl polymerization inhibitor, such as phenothiazine or hydroquinone, for a given time under a pressure sufficient for the material to be heated to be maintained in the liquid phase. In accordance with the examples, the pressure is generally a few bars. According to this process, the esterification residues are converted into monomeric compounds which can be reused subsequently as feedstock for the esterification reaction.

A description is given, in the document GB 923 595, of the production of monomers from the residue from the esterification of acrylic acid with alkanols in the absence of molecular oxygen. It is recommended first to remove, under reduced pressure, all the volatile monomers and then to carry out the heat treatment at a high temperature, between 200 and 350° C., in the presence of sulphuric acid. The monomers resulting from the heat treatment are discharged using a stream of nitrogen. However, the formation of a large amount of coke and the necessary exclusion of oxygen make this process difficult to carry out on the industrial scale.

The document EP 767 163 claims a process for the esterification of acrylic acid by an alcohol during which the heavy product from the bottom of the column for the distillation of the ester is heated at a temperature ranging from 150 to 250° C. in the presence of an acid; the pressure is adjusted so that the decomposition products immediately evaporate; the process is carried out in the presence of oxygen, and an oxygen-comprising extraction gas is led into the bottom product as entertainment agent for the dissociation products.

The document EP 765 861 describes the preparation of butyl acrylate by heat treatment of a mixture of compounds, such as butyl hydroxypropionate and butyl acryloyloxypropionate, in the liquid phase, in the presence of an acid catalyst, such as para-toluene-sulphonic acid or sulphuric acid, and under reduced pressure. The temperature is between 120 and 220° C. and the decomposition products are continuously removed. Up to 30% of water can be added to the mixture, making it possible to improve the yield.

In the document EP 765 860, the heat treatment of the oxyesters formed during the direct esterification of acrylic acid by butanol is preceded by a distillation of the residue comprising the oxyesters. The preliminary distillation is carried out according to conventional temperature and pressure conditions. The heat treatment of the distillate is subsequently carried out in the presence of an acid catalyst under reduced pressure at a temperature of between 150 and 250° C. A stripping gas, comprising oxygen, is used to entrain the decomposition products formed.

The Applicant Company has now developed a process for recovering in value the heavy byproducts generated during the manufacture of butyl acrylate which exhibits the advantage of avoiding the fouling related to the coke formed in the items of equipment used when phenothiazine is used as polymerization inhibitor. The heavy byproducts are decomposed to give recyclable compounds by catalytic cracking according to a flow sheet comprising a stage of distillation of the heavy byproducts followed by a stage of thermal cracking catalysed by sulphuric acid on the light phase from the distillation stage. The problem with the fouling of the plants could in particular be solved by carrying out a distillation at a relatively low temperature and under an inert atmosphere, optionally in the presence of a dispersant, and by optimizing the residual content of phenothiazine in the feed to the cracking stage and by carrying out this cracking stage also under an inert atmosphere.

Without the Applicant Company being committed to any one explanation, it believes that the lowering in the distillation temperature with an inert atmosphere makes it possible to minimize oxidation of the phenothiazine at the source of the formation of coke and of the fouling. The absence of phenothiazine in the distillation top product has in addition proved to be crucial for the catalytic cracking stage.

### SUMMARY OF THE INVENTION

A subject-matter of the present invention is thus a process for the manufacture of C₁-C₄ alkyl (meth)acrylates by a direct esterification of (meth)acrylic acid by the corresponding alcohol in the presence of sulphuric acid, phenothiazine being used as polymerization inhibitor, during which process a bottom product comprising heavy byproducts and phenothiazine is isolated at the bottom of the column for the distillation of the ester formed, characterized in that, in a first stage, the said bottom product is distilled under reduced pressure and under an inert atmosphere and then the distillate resulting from the first stage is subjected to a heat treatment at high temperature at atmospheric pressure under an inert atmosphere and in the presence of sulphuric acid.

In the process according to the invention, the distillate resulting from the first stage comprises a residual phenothiazine content of less than 100 ppm.

The process of the invention can be carried out batchwise or continuously.

Other characteristics and advantages of the invention will more fully emerge on reading the description which follows.

### DETAILED ACCOUNT OF THE INVENTION

Conditions for carrying out the reaction for the direct esterification of (meth)acrylic acid by the corresponding alcohol in the presence of sulphuric acid in order to obtain the C₁-C₄ alkyl (meth)acrylate and the various stages for the purification of the ester formed are described, for example, in the document EP 609 127, incorporated by reference.

In the process of the invention, the bottom product originates from the bottom of the column for the distillation of the C₁-C₄ alkyl (meth)acrylate produced, a final distillation generally being carried out in order to isolate the expected product with high purity.

An average composition of the bottom product, in the case of the manufacture of butyl acrylate in the presence of phenothiazine as polymerization inhibitor, is generally as follows:

| | |
|---|---|
| Butanol: | <0.1% |
| Butyl acrylate: | 5-10% |
| Butyl hydroxypropionate (BHP) | 1-3% |
| Butyl butoxypropionate (BBP) | 70-80% |
| Butyl acryloyloxypropionate (AA/BuA) | 4-6% |
| Dibutyl maleate | 2-5% |
| Phenothiazine | 4-8% |

The bottom product can also comprise other polymerization inhibitors used in combination with phenothiazine, in particular hydroquinone (HQ) and its derivatives, such as hydroquinone methyl ether (HQME), 2,6-di(tert-butyl)-4-methylphenol (BHT) and 2,4-dimethyl-6-(tert-butyl)phenol (Topanol A), salts of thiocarbamic or dithiocarbamic acid, N-oxyl compounds, such as 4-hydroxy-2,2,6,6-tetramethylpiperidine oxyl (4-OH-TEMPO), compounds comprising nitroso groups, such as N-nitrosophenylhydroxylamine and its ammonium salts, quinones, such as benzoquinone, or amino compounds, such as para-phenylenediamine derivatives.

According to the process of the invention, the bottom product is distilled under an inert atmosphere and under reduced pressure.

Use may be made, as inert atmosphere, of a stream of nitrogen or argon, so as to remove the air which may be present in the medium.

The distillation is carried out under a pressure which can range from 133 Pa to 13 300 Pa (1 to 100 mmHg), preferably from 1330 Pa to 6650 Pa (10 to 50 mmHg).

The bottom product is fed to the distillation column at ambient temperature and is brought to a distillation temperature which can range from 50° C. to 150° C.

Preferably, the distillation is carried out at a temperature ranging from 100° C. to 120° C.

The distillation is carried out in a reboiler surmounted by a demister, in order to limit the distillation of the phenothiazine present in the bottom product.

The residence time is suited to the bottom product feed throughput and to the content of phenothiazine present in the distillate. The distillate resulting from the distillation stage comprises less than 100 ppm of phenothiazine, preferably less than 50 ppm.

The bottom product can have a dispersant added to it before being distilled. Mention may in particular be made, as dispersants which can be used, of the phosphoric compounds described in the published international application WO 06/040457, more particularly Beycostat® FB 095. The content of dispersant can vary between 500 and 5000 ppm, more particularly between 1000 and 3000 ppm. The distillate resulting from the distillation stage is composed predominantly of butyl butoxypropionate (BBP), with butyl acrylate and butyl acryloyloxypropionate (AA/BuA).

The distillate is subsequently subjected to a thermal cracking stage at atmospheric pressure. Use is made, for the cracking, of a temperature which can range from 160° C. to 185° C., preferably from 165° C. to 175° C. The thermal cracking is also carried out under an inert atmosphere, which prevents oxidation of the traces of phenothiazine which might be present in the distillate resulting from the first stage.

Use is made of sulphuric acid as catalyst for the thermal cracking, in an amount by weight ranging from 1 to 5% with respect to the feed, more particularly from 3 to 5%. Use may be made of concentrated sulphuric acid, which is introduced into the cracker so as to form the bottoms of the cracker.

The thermal cracking stage can be carried out in the presence of a polymerization inhibitor added to the feed stream of the cracker. Use may be made of conventional polymerization inhibitors other than phenothiazine, such as those mentioned above, in an amount ranging from 200 to 2000 ppm with respect to the feed, preferably from 300 to 1000 ppm.

A dispersant can also be added to the distillate before carrying out the thermal cracking. Mention may be made, as dispersants which can be used, of the same compounds mentioned above for the distillation stage, it being possible for the content of dispersant to vary between 500 and 5000 ppm, more particularly between 1000 and 3000 ppm.

On conclusion of the stage of heat treatment at high temperature, the butyl butoxypropionate (BBP) is decomposed to give butyl acrylate and butanol and the butyl acryloyloxypropionate (AA/BuA) is decomposed to give butyl acrylate and acrylic acid. The decomposition products butanol and acrylic acid are advantageously recycled to the reaction stage. The butyl acrylate thus recovered makes it possible to increase the productive output of the process.

The operating conditions in the process of the invention make it possible to obtain high levels of stripping, expressed as percentages of output throughput with respect to the feed throughput, whether for the distillation stage or thermal cracking stage, which are markedly greater than 60% and which can reach more than 80% during the thermal cracking.

The invention makes it possible to decompose the heavy products generated during the manufacture of C₁-C₄ alkyl (meth)acrylate to give recyclable products while limiting deposits of solid materials in the plants and thus while reducing the frequency of shutdowns necessary for the cleaning of the items of equipment.

The present invention will now be described with the help of examples which do not limit the scope of the invention.

### IMPLEMENTATIONAL EXAMPLES

### Definition

Level of stripping: top product throughput (g/h)/feed throughput (g/h)×100

### Equipment

a) Distillation Stage

Use is made of a glass thermosiphon reboiler with a working capacity of 92 cm³. The reboiler is fed continuously, from a receptacle for BuA heavy products, using a membrane pump equipped with a backpressure valve. The feed stream is conveyed into the reboiler at ambient temperature. The feed throughput is regulated by continuously measuring the weight of mixture in the starting receptacle.

The reboiler is heated using 3 ring heaters (electrical resistances) each with a power of 160 W, so as to reduce as much as possible the skin temperature of the ring heaters. These ring heaters, positioned externally, are brought into contact with the wall of the reboiler through an aluminium foil, the role of which is the diffusion of the heat. A thimble with an external diameter of 10 mm is placed at the centre of the heating region in order to reduce as much as possible the volume of the latter and thus to increase the rate of passage of the heavy products to be distilled. The heating power is adjusted so as to have the desired temperature in the reboiler.

A demisting section, composed of a glass component furnished with a piece of multiknit padding with a height of 3 cm, is added at the top of the reboiler. The vapours exiting from the reboiler are directed to a water-cooled condenser and the distillate is taken up by a membrane pump controlled by a float (level regulation) in order to be directed to a recovery receptacle at atmospheric pressure.

The bottoms are recovered by overflowing in the reboiler and then taken up by a membrane pump controlled by a float (level regulation) in order to be directed to a recovery receptacle at atmospheric pressure.

The temperature measurements are carried out in the leg of the thermosiphon equipped with the ring heaters (thimble), in the side leg of the thermosiphon (internal measurement) and at the top of the demisting section (internal measurement). The operations are carried out under a reduced pressure, generally of 1330 Pa (10 mmHg), regulated using a pressure sensor and an electrically-operated valve.

b) Cracking Stage

The arrangement is identical to that used for the distillation but it does not comprise a demisting section and the cracking reaction is carried out at atmospheric pressure. Use is made of a glass thermosiphon reboiler with a working capacity of 92 cm³. The reboiler is fed continuously using a membrane pump from the receptacle containing the distillate from the preceding stage. The feed stream is conveyed into the cracker at ambient temperature and the pressure is maintained at atmospheric pressure. The feed throughput is regulated by continuously measuring the weight of mixture in the starting receptacle.

The cracker is heated using 3 ring heaters (electrical resistances) with a power of 160 W. These ring heaters, positioned externally, are brought into contact with the wall of the reboiler through an aluminium foil. A thimble with an external diameter of 10 mm is placed at the centre of the heating region in order to reduce as much as possible the volume of the latter and thus to increase the rate of passage of the product to be cracked. The heating power is adjusted so as to have the desired temperature in the reboiler.

The vapours exiting from the cracker are directed to a water-cooled condenser and the distillate is directed to a recovery receptacle at atmospheric pressure.

The bottoms are recovered by overflowing in the reboiler.

The temperature measurements are carried out in the leg of the thermosiphon equipped with the ring heaters (thimble), in the side leg of the thermosiphon (internal measurement) and at the top of the reboiler (outlet for the vapours).

### Example 1

BuA heavy products resulting from a process for the esterification of acrylic acid by butanol in which phenothiazine (PTZ) is used as polymerization inhibitor are distilled.

Before the test, 2000 ppm of dispersant (Beycostat® FB095 from Ceca) are added to the feed.

The distillation is carried out under nitrogen, so as to eliminate the air which might be present in the medium.

The BuA heavy products are fed at ambient temperature and the heating is regulated so as to achieve 117° C. in the reboiler (the temperature is adjusted as a function of the level of stripping desired).

The feed throughput is adjusted so as to have a residence time of 15 minutes.

The quality of the distillate is monitored and it is confirmed in particular that it comprises little PTZ (<100 ppm). A simple and rapid test consists in adding a few drops of sulphuric acid to a flask containing distillate. If the distillate instantaneously darkens, there is too much PTZ. If the distillate has a gradual orangey colouring, the concentration of PTZ is correct. The results of the two tests appear in Table 1 below.

TABLE 1

Test reference

| | Test 1 | Test 2 |
|---|---|---|
| Feed throughput, g/h | 360 | 360 |
| Top product of the reboiler | 236.6 | 255.2 |
| throughput, g/h | | |
| Bottoms of the reboiler | 123.4 | 104.8 |
| throughput, g/h | | |
| Thermosiphon temperature, ° C. | 115.5 | 117.4 |
| Top temperature, ° C. | 107.8 | 109.1 |
| Pressure, Pa (mmHg) | 1330 (10) | 1330 (10) |
| Residence time, min | 15 | 15 |
| Level of stripping, % | 65.7 | 70.9 |

Under these conditions, it is found that the wall of the reboiler remains clean.

### Example 2

The distillate resulting from the distillation stage of Example 1 feeds the cracker continuously and at ambient temperature.

The feed throughput is regulated so as to have a residence time of 30 minutes.

The heating power is adjusted so as to have a temperature in the cracker of the order of 170-175° C.

To initiate the cracking, concentrated sulphuric acid is injected at the start so as to constitute the cracker bottoms (the acid concentrates in the bottoms when stripping of the heavy products begins). 2000 ppm of dispersant (Beycostat® FB 095) and 500 ppm of 4-OH-TEMPO (Nalco) are also added.

The cracking is carried out at atmospheric pressure and under nitrogen (bubbling 71 cm³/min), so as to prevent oxidation of the PTZ traces which might be present in the distillate from the 1^{st} stage.

The results of the two tests appear in Table 2 below.

TABLE 2

Test reference

| | Test 1 | Test 2 |
|---|---|---|
| Feed throughput, g/h | 200 | 200 |
| H₂SO₄ addition, % | 4.3 | 4.3 |
| Beycostat FB 095 addition, ppm | 2029 | 2029 |
| 4-OH-TEMPO addition, % | 0.052 | 0.052 |
| Top product of the cracker | 139 | 177 |
| throughput, g/h | | |
| Bottoms of the cracker throughput, | 61 | 23 |
| g/h | | |
| Thermosiphon temperature, ° C. | 174.3 | 171.3 |
| Top temperature, ° C. | 136 | 135.7 |
| Residence time, min | 30 | 30 |
| Level of stripping, % | 69.5 | 88.5 |

Under these conditions, it is found that the wall of the cracker remains clean.

### Example 3

Table 3 below gives the compositions of the various streams of Test 1, determined by analysis by liquid chromatography (HPLC) and gas chromatography (GC).

TABLE 3

| | | Bottom | | |
|---|---|---|---|---|
| | | product | Distillation | |
| | | feed | top | |
| | | comprising | product | Top |
| | | the BuA | (feed for | product |
| | | heavy | the | of the |
| | Compound | byproducts, % | cracker), % | cracker, % |
| GC | H₂O | 0.021 | 0.046 | 1.95 |
| analysis | Sum of the butenes | | | 7.25 |
| | Butanol | 0.1145 | 0.009 | 7.627 |
| | Sum of the octenes | | | 0.108 |
| | Butyl acetate | | | 0.0407 |
| | Dibutyl ether | | | 8.0830 |
| | BuA | 10.6640 | 16.23 | 68.105 |
| | BBP | 70.464 | 74.625 | 1.902 |
| | AA/BuA | 4.74 | 4.137 | 0.105 |
| HPLC | Acrylic acid | 0.0534 | 0.0350 | 3.51 |
| analysis | AA | | | |
| | HQME | 0.4025 | 0.2735 | 0.0020 |
| | PTZ | 4.07 | 0.0026 | 0.0001 |

1. Process for the manufacture of C₁-C₄ alkyl (meth)acrylates by a direct esterification of (meth)acrylic acid by the corresponding alcohol in the presence of sulphuric acid, phenothiazine being used as polymerization inhibitor, during which process a bottom product comprising heavy byproducts and phenothiazine is isolated at the bottom of the column for the distillation of the ester formed, characterized in that, in a first stage, the said bottom product is distilled under reduced pressure and under an inert atmosphere and then the distillate resulting from the first stage is subjected to a heat treatment at high temperature at atmospheric pressure under an inert atmosphere and in the presence of sulphuric acid.

2. Process according to claim 1, characterized in that it is carried out continuously.

3. Process according to claim 1 or 2, characterized in that the content of phenothiazine in the distillate is less than 100 ppm.

4. Process according to one of claims 1 to 3, characterized in that the distillation is carried out at a temperature ranging from 50 to 150° C.

5. Process according to one of claims 1 to 4, characterized in that the distillation is carried out under an inert atmosphere.

6. Process according to one of claims 1 to 5, characterized in that the distillation is carried out under a pressure ranging from 133 to 13 300 Pa (1 to 100 mmHg).

7. Process according to one of claims 1 to 6, characterized in that the distillation stage and/or the heat treatment is (are) carried out in the presence of a dispersant.

8. Process according to one of claims 1 to 7, characterized in that the alkyl (meth)acrylate is butyl acrylate.
